(19) United States
(12) Patent Application Publication
(10) Pub. No.: US 2025/0025331 A1
(43) Pub. Date: Jan. 23, 2025

(54) METHODS FOR PREPARING COMPOUNDED POLYMER LATEX COMPOSITIONS AND CONDOMS FORMED THEREFROM EXHIBITING IMPROVED MECHANICAL PROPERTIES

(71) Applicant: Church & Dwight Co., Inc., Princeton, NJ (US)

(72) Inventors: Andrew Guttentag, Robbinsville, NJ (US); Caryn Culleton Oryniak, Hillsborough, NJ (US); Timothy Snowden, Howell, NJ (US)

(21) Appl. No.: 18/776,503

(22) Filed: Jul. 18, 2024

Related U.S. Application Data
(60) Provisional application No. 63/527,734, filed on Jul. 19, 2023.

(51) Int. Cl. A61F 6/04; C07K 14/435; C08J 5/24; C08L 9/08

(52) U.S. Cl. CPC A61F 6/04; C07K 14/43518; C08J 5/245; C08L 9/08

(57) ABSTRACT

The present disclosure provides methods for providing elastomeric articles, such as condoms, that exhibit improved mechanical properties. The improved properties are realized by addition of spider silk at specific stages of the method for preparing the compounded polymer latex composition from which the condom is formed.

### CROSS-REFERENCE TO RELATED APPLICATIONS

The present application claims priority to U.S. Provisional App. No. 63/527,734, filed Jul. 19, 2023, the disclosure of which is incorporated herein by reference in its entirety.

### FIELD OF THE DISCLOSURE

The present disclosure relates to methods of preparing compounded polymer latex compositions and products that are formed from the polymer compositions, and the present disclosure more particularly relates to condoms prepared by the described methods. The formed condoms exhibit improved mechanical properties arising from the addition of spider silk at specific stages of the compounding process.

### BACKGROUND

Natural rubber, which is comprised primarily of cis-1,4-polyisoprene, is well known for use in making thin-film, elastomeric articles, such as surgical gloves, condoms, and the like. Various efforts have been made to provide condoms with progressively thinner walls in order to improve the material feel for an enhanced consumer experience. While improving the consumer experience with thinner condoms, the use of thinner walls can increase the potential for breakage during use. Accordingly, there remains a need in the field for thin-film, elastomeric articles, and particularly condoms, that can have thin walls to improve consumer experience while also providing improved strength that enhances consumer confidence in the absence of breakage during use.

### SUMMARY OF THE DISCLOSURE

The present disclosure provides methods of preparing compositions formed of polymeric latex materials that are combined with spider silk (particularly synthetic spider silk), as well as articles made therefrom, and particularly condoms made from the spider silk-modified polymeric latex compositions. Spider silk, both synthetic and natural, is a protein fiber material with an established biocompatibility and is well known for its mechanical properties. Spider silks are often compared in toughness to Kevlar® and in strength to steel cables. It has been found according to the present disclosure that implementation of certain processing conditions when combining spider silk with polymeric latex materials can lead to formation of end products, particularly condoms, that exhibit significantly improved properties over similar articles similarly made without the spider silk additive as well as similar articles made with the spider silk additive but made under different process conditions.

In one or more embodiments, the present disclosure thus may provide a condom formed from a compounded composition prepared by providing a polymer latex dispersion and adding thereto a plurality of compounding ingredients effective to form a compounding formulation; and subjecting the compounding formulation to conditions sufficient to crosslink the polymer latex to a defined cure state and thus provide the composition from which the condom is formed wherein the method further includes combining synthetic spider silk with the polymer latex dispersion prior to forming the compounding formulation or combining the synthetic spider silk with the compounding formulation prior to achieving the defined cure state. In further embodiments, the condom may be defined in relation to one or more of the following statements, which can be combined in any number and order.

The polymer latex can comprise natural rubber latex.

The plurality of compounding ingredients can be selected from the group consisting of stabilizers, surfactants, curing agents, catalysts, accelerators, and antioxidants.

The synthetic spider silk can be combined with the polymer latex dispersion before addition of any curing agents.

The synthetic spider silk can be combined with the polymer latex dispersion before addition of any catalysts.

The synthetic spider silk can be combined with the polymer latex dispersion before addition of any accelerators.

The synthetic spider silk can be combined with the polymer latex dispersion before completing forming the compounding formulation.

The conditions sufficient to crosslink the polymer latex to a defined cure state include heating the compounding formulation above room temperature for a defined length of time.

The synthetic spider silk can be combined with the compounding formulation prior to heating the compounding formulation above room temperature.

The synthetic spider silk is combined with the compounding formulation prior to completion of the defined length of time.

The conditions sufficient to crosslink the polymer latex to a defined cure state can include holding the compounding formulation at one or more temperatures for a total length of time sufficient to achieve the defined cure state.

The synthetic spider silk can be combined with the compounding formulation prior to completion of the defined length of time.

The synthetic spider silk can be provided as a dispersion.

The dispersion has a synthetic spider silk concentration of about 2% to about 20% by weight, based on the total weight of the dispersion.

The composition for forming the condom has a final solids content of about 35% to about 60% by weight, based on the total weight of the composition, including solids attributable to the synthetic spider silk.

In one or more embodiments, a condom can include at least one layer of a film formed from a compounded polymer latex composition that has been crosslinked to a defined cure state and that has synthetic spider silk incorporated therein, wherein the synthetic spider silk is effective to impart improved properties to the condom by having been added to the compounded polymer latex composition before reaching the defined cure state.

The compounded polymer latex composition can comprise natural rubber latex.

The compounded polymer latex composition can comprise a plurality of compounding ingredients selected from the group consisting of stabilizers, surfactants, curing agents, catalysts, accelerators, and antioxidants.

The synthetic spider silk can be effective to impart improved properties to the condom by having been added to the compounded polymer latex composition before subjecting the compounded polymer latex composition to crosslinking conditions.

The synthetic spider silk added to the compounded polymer latex composition can be provided as a dispersion.

The dispersion can have a synthetic spider silk concentration of about 2% to about 20% by weight, based on the total weight of the dispersion.

The compounded polymer latex composition can have a solids content of about 35% to about 60% by weight, based on the total weight of the composition, including solids attributable to the synthetic spider silk.

### BRIEF DESCRIPTION OF THE DRAWINGS

FIG. 1 is a graph showing tensile strength of samples taken from films formed from compounded polymer latex compositions illustrating differences based on the time of addition of synthetic spider silk to the composition.

FIG. 2 is a graph showing average tensile strength for the formulations in the graph of FIG. 1.

FIG. 3 is a graph showing average elongation for the formulations in the graph of FIG. 1.

### DETAILED DESCRIPTION OF THE DISCLOSURE

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present disclosure relates to methods of preparing polymer latex compositions combined with spider silk, methods of forming articles, such as condoms, from the compositions, and articles that are formed at least partially from the compositions. Articles, particularly condoms, that are prepared as described herein can exhibit improved properties relative to similar articles prepared by different methods. Such comparison can, in some embodiments, be relative to substantially identical compositions that are prepared using a different method. Such comparison can, in some embodiments, be relative to compositions that are prepared by substantially the same method but that do not include spider silk.

Elastomeric, thin film articles, such as condoms, are typically prepared using a polymer latex dispersion that is combined with a plurality of compounding ingredients, the result of which can be referenced as being a compounding formulation. The compounding formulation then can be subjected to further processing conditions to achieve a final composition that can be used in methods of article formation, such as dipping methods. According to the present disclosure, addition of spider silk can provide improved properties to the end product (e.g., a condom) if the spider silk is added to the mixture of materials at a defined stage of the method of preparing the composition from which the article is formed. Reference to spider silk herein is understood to encompass the use of natural spider silk as well as synthetic spider silk. In some embodiments, synthetic spider silk can be a preferred choice, and any part of the present disclosure discussing the use of spider silk preferably can mean synthetic spider silk, but such discussion of spider silk likewise may be understood to mean natural spider silk unless expressly stated otherwise. Synthetic spider silk can be prepared via, for example, genetic engineering techniques whereby fibers are created by methods that mimic natural fiber creation and whereby the formed fibers exhibit the traits of natural spider silk while having a completely unique DNA sequence. Synthetic spider silk is available, for example, from Seevix Material Sciences Ltd., Jerusalem Israel.

Polymer compositions made and used in the presently disclosed methods can comprise any suitable polymer composition available in the form a latex (e.g., a polymer latex dispersion). The polymer composition is combined with one or more additives otherwise described herein as compounding ingredients. In some embodiments, suitable polymer latex dispersions may include polymer latex that is natural rubber (“NRL”). In other embodiments, the polymer latex maybe a synthetic rubber latex (“SRL”), or mixtures of NRL and SRL in suitable ratios (e.g., 90/10 to 1/99 NRL to SRL, and more particularly 75/25 to 2/98, 50/50 to 3/97, 40/60 to 4/96, or 30/70 to 5/95 NRL to SRL, the foregoing ratios being weight/weight (“w/w”) ratios). In some embodiments, the use of NRL may additionally expressly exclude the use of any SRL components. Likewise, the polymer latex in a composition according to the preset disclosure may comprise substantially only NRL, which can mean that any polymer latex component other than NRL is present in a total amount of no greater than 0.8% by weight, preferably no greater than 0.5% by weight, and more preferably no greater than 0.1% by weight. Similarly, the polymer latex in a composition according to the preset disclosure may comprise substantially only SRL(s), which can mean that NRL is present in a total amount of no greater than 0.8% by weight, preferably no greater than 0.5% by weight, and more preferably no greater than 0.1% by weight. Non-limiting examples of the types of SRL materials that may be used according to the present disclosure can include polyisoprene, poly (styrene-isoprene-styrene) (“SIS”), intermediate modulus (“IM”) styrene ethylene butylene styrene (“SEBS”), high modulus (“HM”) SEBS, water-based polyurethane, nitrile rubber (e.g., acrylonitrile butadiene rubber, or “NBR”), styrene-co-butadiene, styrene-co-isoprene, triblock copolymers, such as styrene-block-butadiene and block styrene (SBS), and similar, synthetic latex polymers in the form of homopolymers and/or co-polymers may be utilized.

A suitable polymer may be provided in the form of a dispersion in water or other suitable, liquid medium, and may be used as sourced or diluted with water to a desired solids content. In particular, the polymer component(s) can be present in a sufficient amount such that the final polymer composition used for forming the articles has a total solids content of about 30% to about 60%, about 35% to about 58%, about 37% to about 57%, or about 40% to about 55%, based on the total weight of the final composition. Spider silk that is added to the composition can be provided in the form of a dispersion in water or other suitable, liquid medium. A suitable spider silk dispersion can comprise about 1% to about 30%, about 2% to about 20%, or about 3% to about 18% of the spider silk, based on the total weight of the spider silk dispersion. Preferably, a suitable amount of the spider silk dispersion is added to the polymer latex so that the final composition used for forming the articles has a total solids content, including solids attributable to the spider silk, of about 35% to about 60%, about 37% to about 60%, or about 40% to about 60% based on the total weight of the final composition.

In some embodiments, the present disclosure provides condoms formed from a composition that includes spider silk but which is prepared by a specified method to achieve condoms with improved properties that are not achieved if prepared by other methods. In an example embodiment, a preferred method can comprise providing a polymer latex dispersion and adding to the polymer latex dispersion a plurality of compounding ingredients effective to form a compounding formulation. As used herein, a compounding formulation is an intermediate composition that comprises a polymer latex dispersion and an amount of compounding ingredients effective to crosslink the polymer latex when subjected to appropriate processing conditions. Once crosslinking has been carried out to achieve a defined cure state, the combined (and at least partially crosslinked) materials can be referred to as a compounded polymer latex composition.

Compounding ingredients suitable for addition to the polymer latex dispersion can be selected, in some embodiments, from the group consisting of stabilizers, surfactants, curing agents (or sulfur donors), catalysts, accelerators (or cure accelerators), and antioxidants. A suitable group of compounding ingredients can comprise a single material from an individual group (e.g., a single accelerator) or can comprise a plurality of materials from an individual group (e.g., a plurality of accelerators). Further, a suitable group of compounding ingredients can comprise one or more materials from any two or more of the individual groups, any three or more of the individual groups, or any four or more of the individual groups.

In some embodiments, one or more accelerators (or cure accelerators) may be included in the polymer composition. Suitable cure accelerators can include, for example, one or more dithiocarbamates. Non-limiting examples of suitable dithiocarbamates can include zinc dibutyldithiocarbamate (ZDBC), zinc diethydithiocarbamate (ZDEC), zinc dimethyldithiocarbamate (ZDMC), zinc dibenzyl dithiocarbamate (ZBED), sodium diethyl dithiocarbamate (SDEC), and sodium dibutyldithiocarbamate (SDBC).

A single curing accelerator or a mixture of two or more curing accelerators may be used in the polymer composition in a total amount based upon a composition including 100 parts per hundred rubber (phr) of the polymer latex component or combination of polymer latex components. For example, in some embodiments, a single curing accelerator may be used in an amount of about 0.01 to about 5.0 phr, about 0.02 to about 4.0 phr, or about 0.5 to about 3.0 phr. In other embodiments, a single curing accelerator may be used in an amount of about 0.01 to about 2.0 phr, about 0.02 to about 1.8 phr, or about 0.03 to about 1.5 phr. In further embodiments, a total amount of all curing accelerators in the polymer composition can be about 0.1 to about 5.0 phr, about 0.15 to about 4.5 phr, or about 0.2 to about 4.0 phr.

In some embodiments, useful sulfur donors (or curing agents) can include one or more thiurams, such as dipentamethylenethiuram hexasulfide (DPTTH), dipentamethylenethiuram tetrasulfide (DPTT), tetramethylthiuram monosulfide (TMTM), tetramethylthiuram disulfide (TMTD), tetraethylthiuram disulfide (TETD), and tetrabenzylthiuram disulfide (TB2TD). Additionally, or alternatively, other types of sulfur donors may also be utilized. For example, 4,4′-dithiodimorpholine (DTDM), thiocarbamyl sulfonamide, and N-oxydiethylene thiocarbamyl-N-oxydiethylene sulfonamide (OTOS) may be utilized in some embodiments. The use of such materials can be beneficial in that the sulfur included in the sulfur donor compounds is not free sulfur that can contribute to potential allergies. If desired, however, free sulfur can be used, such as any source of soluble sulfur, amorphous sulfur, and derivatives thereof. Likewise, multiple sulfur donors may be used and may be provided as a sulfur dispersion.

A single curing agent/sulfur donor or a mixture of two or more curing agents/sulfur donors may be used in the polymer composition in a total amount based upon a composition including 100 parts per hundred rubber (phr) of the polymer latex component or combination of polymer latex components. For example, in some embodiments, a single curing agent/sulfur donor may be used in an amount of about 0.01 to about 5.0 phr, about 0.02 to about 4.0 phr, or about 0.5 to about 3.0 phr. In other embodiments, a single curing agent/sulfur donor may be used in an amount of about 0.01 to about 2.0 phr, about 0.02 to about 1.8 phr, or about 0.03 to about 1.5 phr. In further embodiments, a total amount of all curing agents/sulfur donors in the polymer composition can be about 0.1 to about 5.0 phr, about 0.5 to about 4.5 phr, or about 1.0 to about 4.0 phr.

In some embodiments, useful surfactants can include any single material or combinations of one or more materials selected from the group cationic surfactants, anionic surfactants, and amphoteric surfactants. Non-limiting examples of suitable surfactants include sodium dodecyl sulfate, sodium lauryl sulfate, sodium polynaphthalene sulfonate, sodium polymethacrylate, monosodium N-lauryl-beta-iminodipropionic acid, potassium laurate, and polyethylene oxides. In some embodiments, useful catalysts (or activators) can include one or more metal oxides. For example, suitable metal oxides can include zinc oxide, magnesium oxide, and other metal oxides with similar activities.

In some embodiments, useful antioxidants can include any material useful to limit non-desired oxidation reactions. Non-limiting examples include polyphenols and mercapto-imidazole compounds, such as 2-mercaptobenzimidazole (MBI), 2-mercaptotoluimidazole (MTT), 2-mercapto toluimidazole (MTI), a zinc salt of 2-mercaptobenzimidazole (ZMBI), a zinc salt mercaptotoluimidazole (ZNTI), and the like. A further example of suitable antioxidants include butylated reaction products of p-cresol and dicylopentadiene, such as that is available under the name Bostex™ 24.

In some embodiments, useful stabilizers can include various amines, and particularly aromatic amines, as well as caseins, such as ammonium caseinate. Likewise, some materials useful as antioxidants can also be used as stabilizers, such as heterocyclic and phenolic antioxidants.

Catalysts/activators (individually or in total), surfactants (individually or in total), antioxidants (individually or in total), and stabilizers (individually or in total), may independently be included in an amount of about 0.01 to about 3.0 phr, about 0.02 to about 2.0 phr, about 0.03 to about 1.5 phr, or about 0.05 to about 1.2 phr.

Methods of preparing elastomeric articles, particularly condoms, according to the present disclosure can comprise a plurality of steps. In some embodiments, the methods can include any one or more of providing, combining, and mixing of components suitable to form a compounded polymer latex composition. In some embodiments, the methods can include one or more steps wherein a former of other mold is dipped or otherwise coated with one or more coatings or layers of a compounded polymer latex composition to form a film of a desired thickness. In some embodiments, the methods can comprise one or more curing steps wherein a film formed by dipping a former or other mold in a compounded polymer latex composition is processed to be in a substantially finished form (e.g., crosslinked or otherwise solidified to form a unitary article of manufacture). Optionally, one or more drying steps may be utilized. The present disclosure may comprise only the steps of providing, combining, and mixing of components to form the compounded polymer latex composition. The present disclosure may comprise the providing, combining, and mixing steps in combination with the one or more steps wherein a former or other mold is dipped or otherwise coated to form the film, optionally also including the one or more curing steps and the one or more drying steps. The present disclosure may comprise using a pre-formed, compounded polymer latex composition for carrying out the one or more steps wherein a former or other mold is dipped or otherwise coated to form the film, optionally also including the one or more curing steps and the one or more drying steps. Further, suitable processing equipment may be used as needed to provide for the necessary processing steps, including formers, dip tanks, heating equipment, fans, conveyers, and the like.

In one or more embodiments, a method for preparing a condom according to the present disclosure may comprise providing a polymer latex dispersion and adding thereto a plurality of compounding ingredients effective to form a compounding formulation. The polymer latex dispersion may be provided in a pre-formed dispersion, or solid polymer latex (e.g., particles) may be obtained and formed into a dispersion for use in the presently disclosed methods. The polymer latex utilized may be any single polymer material described herein or any combination of two or more polymer materials described herein or two or more polymers included in any one or more classes of polymer materials described herein. The compounding ingredients may be chosen to include any combination of ingredients described herein that is effective to form a compounded polymer latex composition suitable for dip molding or similar method of forming a condom or like article of manufacture. In some embodiments, the plurality of compounding ingredients can comprise at least one or more curing agents and one or more accelerators. In some embodiments, the plurality of compounding ingredients can comprise at least one or more curing agents, one or more accelerators, and one or more surfactants. In some embodiments, the plurality of compounding ingredients can comprise at least one or more curing agents, one or more accelerators, and one or more catalysts. In some embodiments, the plurality of compounding ingredients can comprise at least one or more curing agents, one or more accelerators, one or more surfactants, and one or more catalysts. In some embodiments, one or more stabilizers and/or one or more antioxidants can be included in any of the foregoing example embodiments. In further example embodiments, curing agents can be used in a total amount of about 0.5 to about 2.5 phr, accelerators can be used in a total amount of about 0.1 to about 1.0 phr, surfactants (when present) can be used in a total amount of about 0.02 to about 1.5 phr, catalysts (when present) can be used in a total amount of about 0.2 to about 2.0 phr, stabilizers (when present) can be used in a total amount of about 0.01 to about 0.5 phr, and antioxidants (when present) can be used in a total amount of about 0.05 to about 1.5 phr.

The methods further may include subjecting the compounding formulation to conditions sufficient to crosslink the polymer latex to a defined cure state and thus provide the composition from which the condom is formed. Compounding of the polymer latex and the added compounding ingredients can include at least mixing of the plurality of compounding ingredients with the polymer latex dispersion. The mixing can be carried out at substantially ambient temperature; however, compounding to achieve a defined cure state can be hastened at increased temperatures. If ambient temperature is not sufficiently high, mixing can be carried out with added heating. Preferably, mixing is carried out at temperatures in the range of about 25° C. to about 60° C., about 30° C. to about 58° C., or about 35° C. to about 55° C. In some embodiments, added heat is increased over a period of time during addition of the compounding ingredients, after all compounding ingredients have been added, or both during and after adding the compounding ingredients. A maximum temperature achieved during compounding can be about 40° C. to about 60° C. or about 45° C. to about 55° C. Temperatures used during crosslinking of the polymer latex with the compounding ingredients and duration of compounding to achieve desired crosslinking and thus the defined cure state can vary, and it is expected that time will be higher when lower temperatures are used, and time will be lower when higher temperatures are used. When adding heat up to the noted maximums, the defined cure state may be achieved in a time of about 6 hours to about 24 hours, about 8 hours to about 22 hours, or about 12 hours to about 20 hours. When lower heat or no added heating is used, the defined cure state may be achieved in a time of about 12 hours to about 48 hours, about 18 hours to about 40 hours, or about 24 hours to about 36 hours. When antioxidants are used, in some embodiments, it can be preferred to add antioxidants after any heating has been applied. Further, once the defined cure state has been achieved, water may be added to bring the compounded latex composition to the desired, final solids concentration.

Identifying when the polymer latex has reached the necessary, defined cure state can be done using any industry-recognized standard. As a non-limiting example, vulcanization processes as described herein for providing crosslinked polymer latex compositions can be monitored through a chloroform coagulation test to identify the cure state at a given point of the process and determine if the curing has yet reached the defined, or desired state. The chloroform coagulation test is a qualitative evaluation of cure state in natural rubber latex through solvent-induced coagulation. To carry out the test, equal volumes of chloroform and a sample from the latex dispersion are mixed together to induce coagulation. The resulting, coagulated latex can be visually and tactilely inspected for cure state by comparing to established vulcanization progress benchmarks, such as a numerical scale where the assigned number corresponds to a relative cure state (e.g., where lower values indicate less cure and higher values indicate more cure). A scale from 1 to 4, as an example, is sometimes used. In such example, each rating can correspond to a physical state of the mixture that defines the degree of curing or vulcanization as follows: (1) the latex/chloroform mixture is characterized by forming a tacky lump that is stringy when broken apart; (2) the latex/chloroform mixture is characterized by forming tender lumps that beak apart with short extensions instead of long strings; (3) the latex/chloroform mixture is characterized by forming non-tacky agglomerates; and (4) the latex/chloroform mixture is characterized by forming small, dry crumbs. The exact cure state is based on the desired mechanical properties of the natural rubber latex for the target application. See, for example, Myint, N N; Aye, T T; Naing, K M; Wynn, N. Performance Study of the Natural Rubber Composite with Clay Minerals. Jour. Myan. Acad. Arts & Sc. 2008 6(1), 151-159, the disclosure of which is incorporated by reference herein. For condoms, typically a chloroform test number of (3) is the desired cure state for the prepared latex dispersion; however, a defined cure state as described herein may have a higher test number or a lower test number, or may be evaluated using a different test method with a different scale.

In addition to the foregoing, methods for preparing a condom according to the present disclosure include combining spider silk with the polymer latex. As shown in the appended Examples, it has been found according to the present disclosure that order of addition of the spider silk during the methods of preparing the compounded latex composition has a significant effect on the physical properties of the articles (e.g., condoms) formed with the compounded polymer latex composition. In particular, in various embodiments, it can be preferable to combine the spider silk with the polymer latex dispersion prior to forming the compounding formulation. This can include adding the spider silk to the polymer latex dispersion before addition of any of the compounding ingredients or before addition of any compounding ingredients directly responsible for crosslinking of the polymer latex. In an example embodiment, the spider silk is combined with the polymer latex dispersion before addition of any curing agents. In an example embodiment, the spider silk is combined with the polymer latex dispersion before addition of any catalysts. In an example embodiment, the spider silk is combined with the polymer latex dispersion before addition of any accelerators. In an example embodiment, the spider silk is combined with the polymer latex dispersion before completing forming the compounding formulation (i.e., the final compounding formulation that is in condition for use in dip molding of condoms or the like). In some embodiments, it can be preferable to combine the spider silk with the compounding formulation prior to achieving the defined cure state.

As discussed above, providing conditions that are sufficient to crosslink the polymer latex to a defined cure state can include heating the compounding formulation above room temperature for a defined length of time. In some embodiments, the spider silk can be combined with the compounding formulation prior to adding heating effective to heat the compounding formulation above room temperature. In some embodiments, the spider silk can be combined with the compounding formulation at any point prior to completion of the defined length of time that is utilized to achieve the defined cure state for the compounded polymer latex composition. In certain embodiments, the conditions sufficient to crosslink the polymer latex to a defined cure state can include holding the compounding formulation at one or more temperatures for a total length of time sufficient to achieve the defined cure state. As such, the spider silk can be combined with the compounding formulation prior to completion of the defined total length of time that is sufficient to achieve the defined cure state.

The above conditions for addition of the spider silk to the polymer latex dispersion are believed to be critical for achieving improved physical properties to the finally formed articles (e.g., condoms). Without wishing to be bound by theory, it is believed that addition of the spider silk during crosslinking leads to incorporation of the spider silk into the crosslinked polymer backbone structure in a manner that physically incorporates the mechanical properties of the spider silk into the polymer backbone structure. While the spider silk itself is known to exhibit high strength and durability, it was surprising to find that the spider silk physical properties did not manifest themselves in the polymer latex composition when introduced after crosslinking. Rather, it is only when incorporated into the crosslinked backbone structure of the compounded polymer latex that the superior mechanical properties of the spider silk are manifested in the articles formed from the compounded polymer latex compositions.

The present disclosure particularly can provide elastomeric articles, specifically condoms, that are formed from compounded polymer latex compositions that include spider silk as described above. In some embodiments, the present disclosure thus can provide a condom comprising at least one layer of a film formed from a compounded polymer latex composition that has been crosslinked to a defined cure state and that has synthetic spider silk incorporated therein, wherein the synthetic spider silk is effective to impart improved properties to the condom by having been added to the compounded polymer latex composition before reaching the defined cure state. Such condoms can otherwise be defined in relation to any element of the foregoing disclosure, including the specific polymer(s) used in the compounded polymer latex composition, the compounding ingredients that are utilized, and the order of addition of the spider silk relative to the compounding ingredients and/or the curing conditions, and/or the level of cure state of the compounded polymer latex composition.

In some embodiments, the compounded polymer latex composition used to form the condom can comprise natural rubber latex alone or in combination with at least one synthetic rubber latex. In some embodiments, the compounded polymer latex composition from which the condom is formed can comprise a plurality of compounding ingredients selected from the group consisting of stabilizers, surfactants, curing agents, catalysts, accelerators, and antioxidants. In some embodiments, the spider silk is effective to impart improved properties to the condom by having been added to the compounded polymer latex composition before subjecting the compounded polymer latex composition to crosslinking conditions. In some embodiments, the spider silk added to the compounded polymer latex composition is provided as a dispersion. Improved properties of condoms incorporating spider silk as described herein may be defined, in some embodiments, relative to the tensile strength of the condom. In example embodiments, a condom formed from a compounded polymer latex composition prepared according to the present disclosure can exhibit an increase in tensile strength of at least 20% relative to a condom that does not have spider silk included therein but is otherwise identical in composition. In further embodiments, the increase in tensile strength can be at least 25%, at least 30%, at least 40%, or at least 50% (optionally having a maximum increase of about 100%). Tensile strength can be as evaluated according to ASTM D412.

Physical properties of the condoms that are produced according to the present disclosure may relate to the average thickness of the condom. The presently disclosed compositions may be particularly useful in forming relatively thin-walled structures that still exhibit the overall strength (e.g., at least a minimum tensile strength) that is desired. In one or more embodiments, physical characteristics defined herein may relate to a condom having an average thickness of less than 0.1 mm, less than 0.09 mm, or less than 0.08 mm (e.g., down to a minimum thickness of about 0.01 mm). Preferably, the condoms may have a thickness of about 0.04 mm to about 0.09 mm, about 0.045 mm to about 0.085 mm, or about 0.06 mm to about 0.08 mm.

### Example

### Evaluation of Condom Formulation Tensile Strength

The ability to increase tensile strength without decreasing elongation was evaluated in a natural rubber latex compounding process using a liquid dispersion by comparing the impact of the addition of synthetic spider silk (Seevix) during compounding to a post-compounding addition of synthetic spider silk. The compounding base formulation was created by adding ingredients in a specified amount measured in part per hundred rubber (phr) to form a natural rubber latex dispersion, which was mixed while heating. The ingredients used for both compositions (i.e., Formulation 1, where the spider silk was added after compounding, and Formulation 2, where the spider silk was added during the compounding steps of the process) are shown in TABLE 1.

TABLE 1

| Order | Ingredient | Purpose |
|---|---|---|
| 1 | Natural Rubber Latex | Latex |
| 2 | Ammonium Caseinate | Stabilizer |
| 3 | Sodium Polynaphthalene Sulfonate | Surfactant |
| 4 | Sulfur Dispersion | Curing Agent |
| 5 | Zinc Oxide Dispersion | Catalyst |
| 6 | Dithiocarbamate blend | Accelerator |
| 7 | Sodium dibutyldithiocarbamate | Accelerator |
| 8 | Polyphenol Antioxidant | Antioxidant |
| 9 | DI Water | Solvent |

Following the addition of natural rubber latex, each of the noted compounding ingredients was added in the order listed. At the last step a solvent, DI water, was added to the compounding formulation. Temperature was during steps 1 to 7 to a final temperature of 50° C. and held at that temperature for an additional 16 hours. The temperature was then reduced to room temperature before the addition of the antioxidant at step 8 and prior to dilution with water to hit a target solids percentage of 50%.

The impact of when the synthetic spider silk was added into the formulation was evaluated based on the order of addition shown in TABLE 2. For the preparation of Formulation 1, the synthetic spider silk was added at a concentration of 5% (w/w) based on the dry solids amount, after the antioxidant and when the heating was complete, which is considered a post-addition after compounding. For the preparation of Formulation 2, the synthetic spider silk was added at a concentration of 5% (w/w) based on the dry solids amount after the surfactant, which is a dispersing and stabilizing agent, but still during the time when the heat is contributing to the crosslinking of the natural rubber latex. Each formulation was prepared to a target solids concentration of 50% by weight, including the solids contribution of the synthetic spider silk.

TABLE 2

| Order | Formulation 1 | Formulation 2 |
|---|---|---|
| 1 | Natural Rubber Latex | Natural Rubber Latex |
| 2 | Ammonium Caseinate | Ammonium Caseinate |
| 3 | Sodium Polynaphthalene Sulfonate | Sodium Polynaphthalene Sulfonate |
| 4 | Sulfur Dispersion | Synthetic Spider Silk Dispersion |
| 5 | Zinc Oxide Dispersion | Sulphur Dispersion |
| 6 | Dithiocarbamate blend | Zinc Oxide Dispersion |
| 7 | Sodium dibutyldithiocarbamate | Dibutyldithiocarbamate blend |
| 8 | Polyphenol Antioxidant | Sodium dibutyldithiocarbamate |
| 9 | Synthetic Spider Silk Dispersion | Polyphenol Antioxidant |
| 10 | DI Water | DI Water |

Once each formulation had reached the defined cure state, mechanical properties were evaluated. Thin films of approximately 100 microns thick were created using a drawdown on a glass plate and then heating in an oven at 90° C. for 20 minutes. The films were covered with a corn starch slurry that was added during the removal process to prevent the films from sticking to themselves when being removed from the glass plate.

At least five strips were cut from each film and tested for tensile strength and elongation. Each strip was prepared to a total length of about 80 mm and a total width of about 6 mm. Testing was done following guidelines outlined in ASTM D412: Standard Test Methods for Vulcanized Rubber and Thermoplastics Elastomers-Tension. A Lloyd Ametek LS5 Digital Material Tester with a 50N load cell, speed of 500 mm/min, and 30 mm distance between grips was used during testing. Like testing was carried out on strips prepared from a control formulation, which did not include any added spider silk but was otherwise substantially identical to the test formulations.

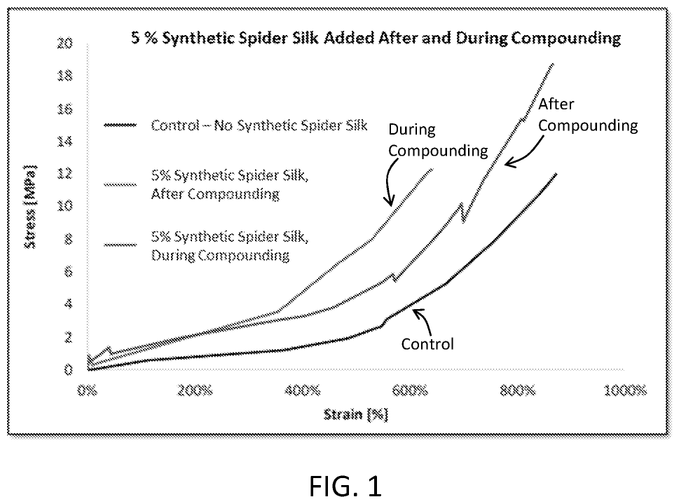

Stress-strain curves for the test formulations and the control formulation were prepared based on the test results. Test data is shown in TABLE 3, and the data is also shown in the graph shown in FIG. 1 Formulation 1, where the synthetic spider silk was added after compounding, showed no improvement in elongation. The samples of formulation 1 also showed a reduction in elasticity, and the samples of formulation 1 were stiffer in comparison to the control. Formulation 2, where the synthetic spider silk added during compounding, showed improved mechanical properties with an increased tensile strength and did not exhibit any loss of elongation in comparison to the control.

TABLE 3

| | | | 5% Synthetic Spider | | 5% Synthetic Spider | |
|---|---|---|---|---|---|---|
| | | | Silk Added After | | Silk Added During | |
| | Control | | Compounding | | Compounding | |
| | | St. | | St. | | St. |
| Parameter | Avg. | Dev. | Avg. | Dev. | Avg | Dev. |
| Tensile | 12 | 1.4 | 13.8 | 1.3 | 18.2 | 2.4 |
| Strength (MPa) | | | | | | |
| Elongation (%) | 831 | 126 | 668 | 39 | 840 | 33 |

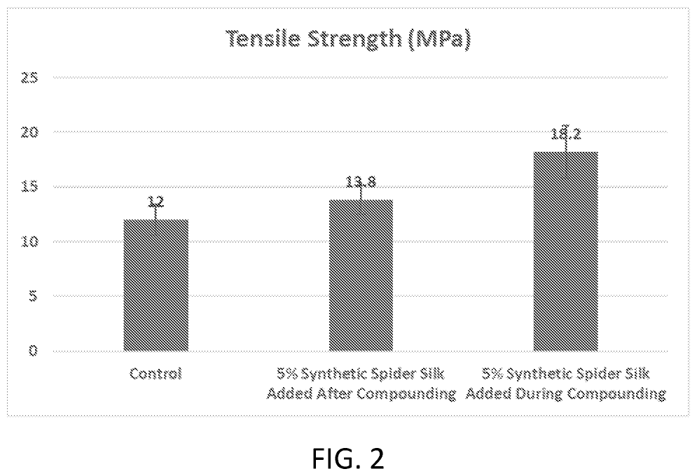

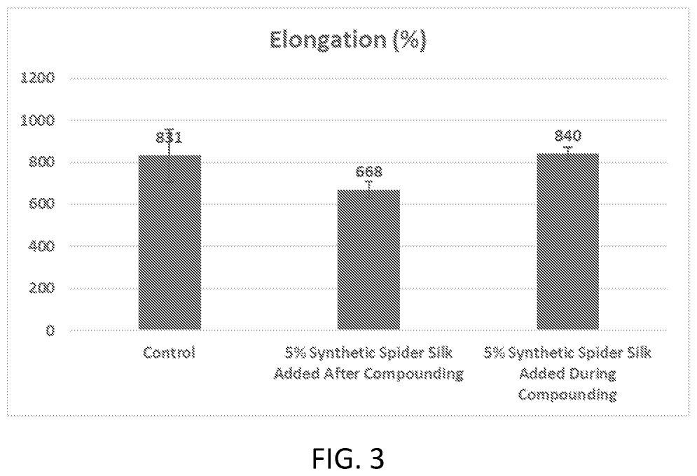

Tensile strength and elongation are further illustrated in FIG. 2 and FIG. 3, respectively. With reference to FIG. 2, increasing average tensile strength was observed for both test formulations, but there was significant overlap in the error bars between the control formulation and formulation 1 where the synthetic spider silk was added after compounding, and the difference in tensile strength between the control and formulation 1 was within the measurement error. With reference to FIG. 3, the elongation parameter was observed to decrease for formulation 1 where the synthetic spider silk was added after the compounding process but remained unchanged for formulation 2 where the addition was done during compounding.

Relative to the control formulation, the method according to the present disclosure whereby the synthetic spider silk was added during compounding and before achieving the defined cure state resulted in an article with a 52% increase in tensile strength and a 1% increase in elongation. On the other hand, utilizing a method whereby the synthetic spider silk was added after the compounding process resulted in a tensile strength increase of 15% along with a corresponding 20% decrease in elongation.

The terms “about”, “substantially”, and “generally” as used herein can indicate that certain recited values or conditions are intended to be read as encompassing the expressly recited value or condition and also values that are relatively close thereto or conditions that are recognized as being relatively close thereto. For example, unless otherwise indicated herein, a value of “about” a certain number or “substantially” or “generally” a certain value or result can indicate the specific number, value, or result as well as numbers, values, or results that vary therefrom (+ or −) 2% or less, or 1% or less. Similarly, unless otherwise indicated herein, a condition that substantially exists can indicate the condition is met exactly as described or claimed or is within typical manufacturing tolerances or would appear to meet the required condition upon casual observation even if not perfectly meeting the required condition. In some embodiments, the values or conditions may be defined as being express and, as such, the term “about” or “substantially” (and thus the noted variances) may be excluded from the express value. Where a plurality of possible lower end values and a plurality of possible upper end values are provided for a particular parameter, it is understood that all possible combinations of values inclusive of any of the lower end values and any of the upper end values are encompassed for describing the parameter.

Many modifications and other embodiments of the disclosure will come to mind to one skilled in the art to which this disclosure pertains having the benefit of the teachings presented in the foregoing description; and it will be apparent to those skilled in the art that variations and modifications of the present disclosure can be made without departing from the scope or spirit of the disclosure. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A condom formed from a composition prepared by a method comprising:
providing a polymer latex dispersion and adding thereto a plurality of compounding ingredients effective to form a compounding formulation; and
subjecting the compounding formulation to conditions sufficient to crosslink the polymer latex to a defined cure state and thus provide the composition from which the condom is formed;
wherein the method further comprises combining synthetic spider silk with the polymer latex dispersion prior to forming the compounding formulation or combining the synthetic spider silk with the compounding formulation prior to achieving the defined cure state.

2. The condom formed from a composition prepared by the method of claim 1, wherein the polymer latex comprises natural rubber latex.

3. The condom formed from a composition prepared by the method of claim 1, wherein the plurality of compounding ingredients are selected from the group consisting of stabilizers, surfactants, curing agents, catalysts, accelerators, and antioxidants.

4. The condom formed from a composition prepared by the method of claim 1, wherein the synthetic spider silk is combined with the polymer latex dispersion before addition of any curing agents.

5. The condom formed from a composition prepared by the method of claim 1, wherein the synthetic spider silk is combined with the polymer latex dispersion before addition of any catalysts.

6. The condom formed from a composition prepared by the method of claim 1, wherein the synthetic spider silk is combined with the polymer latex dispersion before addition of any accelerators.

7. The condom formed from a composition prepared by the method of claim 1, wherein the synthetic spider silk is combined with the polymer latex dispersion before completing forming the compounding formulation.

8. The condom formed from a composition prepared by the method of claim 1, wherein the conditions sufficient to crosslink the polymer latex to a defined cure state include heating the compounding formulation above room temperature for a defined length of time.

9. The condom formed from a composition prepared by the method of claim 8, wherein the synthetic spider silk is combined with the compounding formulation prior to heating the compounding formulation above room temperature.

10. The condom formed from a composition prepared by the method of claim 8, wherein the synthetic spider silk is combined with the compounding formulation prior to completion of the defined length of time.

11. The condom formed from a composition prepared by the method of claim 1, wherein the conditions sufficient to crosslink the polymer latex to a defined cure state include holding the compounding formulation at one or more temperatures for a total length of time sufficient to achieve the defined cure state.

12. The condom formed from a composition prepared by the method of claim 11, wherein the synthetic spider silk is combined with the compounding formulation prior to completion of the defined length of time.

13. The condom formed from a composition prepared by the method of claim 1, wherein the synthetic spider silk is provided as a dispersion.

14. The condom formed from a composition prepared by the method of claim 13, wherein the dispersion has a synthetic spider silk concentration of about 2% to about 20% by weight, based on the total weight of the dispersion.

15. The condom formed from a composition prepared by the method of claim 1, wherein the composition for forming the condom has a final solids content of about 35% to about 60% by weight, based on the total weight of the composition, including solids attributable to the synthetic spider silk.

16. A condom comprising:
at least one layer of a film formed from a compounded polymer latex composition that has been crosslinked to a defined cure state and that has synthetic spider silk incorporated therein,
wherein the synthetic spider silk is effective to impart improved properties to the condom by having been added to the compounded polymer latex composition before reaching the defined cure state.

17. The condom of claim 16, wherein the compounded polymer latex composition comprises natural rubber latex.

18. The condom of claim 16, wherein the compounded polymer latex composition comprises a plurality of compounding ingredients selected from the group consisting of stabilizers, surfactants, curing agents, catalysts, accelerators, and antioxidants.

19. The condom of claim 16, wherein the synthetic spider silk is effective to impart improved properties to the condom by having been added to the compounded polymer latex composition before subjecting the compounded polymer latex composition to crosslinking conditions.

20. The condom of claim 16, wherein the synthetic spider silk added to the compounded polymer latex composition is provided as a dispersion.

21. The condom of claim 20, wherein the dispersion has a synthetic spider silk concentration of about 2% to about 20% by weight, based on the total weight of the dispersion.

22. The condom of claim 16, wherein the compounded polymer latex composition has a solids content of about 35% to about 60% by weight, based on the total weight of the composition, including solids attributable to the synthetic spider silk.
